(19) United States
(12) Patent Application Publication
(10) Pub. No.: US 2025/0259716 A1
(43) Pub. Date: Aug. 14, 2025

(54) METAMATERIAL DESIGN ECOSYSTEM USING PHYSICS MODELING AND MACHINE LEARNING

(71) Applicant: Hamilton Sundstrand Corporation, Charlotte, NC (US)

(72) Inventors: Soumalya Sarkar, Manchester, CT (US); Andrew McClung, Florence, MA (US)

(21) Appl. No.: 18/439,967

(22) Filed: Feb. 13, 2024

(51) Int. Cl. G16C 60/00; G06F 30/27; G06F 111/04; G06F 119/08

(52) U.S. Cl. CPC G16C 60/00; G06F 30/27; G06F 2111/04; G06F 2119/08

(57) ABSTRACT

A computer assisted design ecosystem for materials design includes a computer system including a memory and a processor. The memory stores a computer aided design ecosystem including a physics analysis module and a machine learning analysis module. The physics module includes a physics based reflectance simulation and a ratio of solar absorptivity to thermal emissivity module. The machine learning module includes a machine learning based surrogate model, a compute-aware Bayesian sampling module, and a machine learning metamaterial representation. The memory and processor are configured to utilize the machine learning module and the physics module to iteratively determine a suitable metamaterial structure and composition in response to receiving at least one set of design constraints.

### BACKGROUND

Exemplary embodiments pertain to the art of materials design and selection for space-based applications, and more particularly to a design procedure for creating material structures having suitable heat dissipation qualities.

Designing materials for space-based applications involves multiple factors that are not as impactful for terrestrial based applications. One such factor is related to heat dissipation via emissivity. As space-based objects are not within an atmosphere to help dissipate heat, the heat dissipation of any on-board components is complicated. In particular, heat dissipation surfaces should be designed in such a manner that the surface can radiate heat, while simultaneously minimizing sunlight absorption. This balancing act allows for the electronics in space-based applications to be maintained at the lowest possible temperature, which in turn improves performance of the electronics.

### BRIEF DESCRIPTION

Disclosed is a computer assisted design ecosystem for materials design includes a computer system including a memory and a processor. The memory stores a computer aided design ecosystem including a physics analysis module and a machine learning analysis module. The physics module includes a physics based reflectance simulation and a ratio of solar absorptivity to thermal emissivity module. The machine learning module includes a machine learning based surrogate model, a compute-aware Bayesian sampling module, and a machine learning metamaterial representation. The memory and processor are configured to utilize the machine learning module and the physics module to iteratively determine a suitable metamaterial structure and composition in response to receiving at least one set of design constraints.

In another example, the machine learning module is configured to receive the metamaterial representation and a calculated ratio of solar absorptivity to thermal emissivity, determine an adaptive sampling set, and provide the adaptive sampling set to the compute-aware Bayesian sampling module.

In another example of any of the above, the compute-aware Bayesian sampling module is configured to receive an adaptive sampling set, generate a machine learning representation of an expected suitable metamaterial design space based on the adaptive sampling set, and provide the machine learning representation of the expected suitable metamaterial design space to the physics module.

In another example of any of the above, the physics based simulator is configured to identify an example metamaterial structure and composition using a machine learning representation of an expected suitable metamaterial design space from the machine learning metamaterial representation and determine a reflectivity and an absorptivity of the example metamaterial structure.

In another example of any of the above a ratio of solar absorptivity to thermal emissivity module of the example metamaterial structure and composition is determined using the ratio of solar absorptivity to thermal emissivity module, and wherein the ratio of solar absorptivity to thermal emissivity is provided to the multi-source surrogate modeling module.

In another example of any of the above the computer system is further configured to output the suitable metamaterial structure and composition to a manufacturing system, and wherein the manufacturing system is configured to create the suitable metamaterial structure.

In another example of any of the above the suitable metamaterial structure is a heat dissipation surface for a space-based unmanned component.

In another example of any of the above utilizing the machine learning module and the physics module to iteratively determine a suitable metamaterial structure and composition in response to receiving at least one set of design constraints comprises iteratively determining a single most suitable metamaterial structure and composition.

In another example of any of the above utilizing the machine learning module and the physics module to iteratively determine a suitable metamaterial structure and composition in response to receiving at least one set of design constraints comprises iteratively determining a plurality of suitable metamaterial structures and compositions, and wherein the machine learning based surrogate model is configured to output the plurality of suitable metamaterial structures and compositions to a material designer.

Also disclosed is a method for generating a heat dissipation surface material for a space based application, the method including iteratively determining a suitable metamaterial structure and composition in response to receiving at least one set of design constraints using a computer based design ecosystem. The computer-based design ecosystem includes a physics module having a physics based reflectance simulation and a ratio of solar absorptivity to thermal emissivity module and a machine learning module including a machine learning based surrogate model, a compute-aware Bayesian sampling module, and a machine learning metamaterial representation.

In another example of the above method, receiving the metamaterial representation and a calculated ratio of solar absorptivity to thermal emissivity at the machine learning module, determining an adaptive sampling set using the machine learning based surrogate model, and providing the adaptive sampling set to the compute-aware Bayesian sampling module.

In another example of any of the above methods, the method further includes receiving an adaptive sampling set at the Bayesian sampling module, generate a machine learning representation of an expected suitable metamaterial design space based on the adaptive sampling set using the Bayesian sampling module, and providing the machine learning representation of the expected suitable metamaterial design space from the Bayesian sampling module to the physics module.

In another example of any of the above methods, the method further includes identifying an example metamaterial structure and composition using a machine learning representation of an expected suitable metamaterial design space from the machine learning metamaterial representation and determining a reflectivity and an absorption rate of the example metamaterial structure using the physics based reflectance simulation.

In another example of any of the above methods, the method further includes determining a ratio of solar absorptivity to thermal emissivity module of the example metamaterial structure and composition using the ratio of solar absorptivity to thermal emissivity module, and providing the ratio of solar absorptivity to thermal emissivity to the multi-source surrogate modeling module.

In another example of any of the above methods, the method further includes outputting the suitable metamaterial structure and composition to a manufacturing system and creating the suitable metamaterial structure.

In another example of any of the above methods, the method further includes constructing a space-based unmanned component including at least one heat dissipation surface comprising the suitable metamaterial structure.

In another example of any of the above methods, iteratively determining a suitable metamaterial structure and composition in response to receiving at least one set of design constraints comprises iteratively determining a single most suitable metamaterial structure and composition.

In another example of any of the above methods iteratively determining a suitable metamaterial structure and composition in response to receiving at least one set of design constraints comprises iteratively determining a plurality of suitable metamaterial structures and compositions and outputting the determined plurality of suitable metamaterial structures to a materials designer.

### BRIEF DESCRIPTION OF THE DRAWINGS

The following descriptions should not be considered limiting in any way. With reference to the accompanying drawings, like elements are numbered alike:

FIG. 1 is a schematic representation of an exemplary unmanned space-based device.

FIG. 2 illustrates an exemplary pair of image outputs showing an impact temperature has on an operation of a space-based component.

FIG. 3 is a schematic representation of a metamaterial for heat dissipation in a space-based application.

FIG. 4 illustrates a Machine learning (ML)-guided design ecosystem for designing a metamaterial for heat dissipation.

FIG. 5 schematically illustrates an application of the design ecosystem of FIG. 4 applied to two five-layer metamaterial designs.

### DETAILED DESCRIPTION

A detailed description of one or more embodiments of the disclosed apparatus and method are presented herein by way of exemplification and not limitation with reference to the Figures.

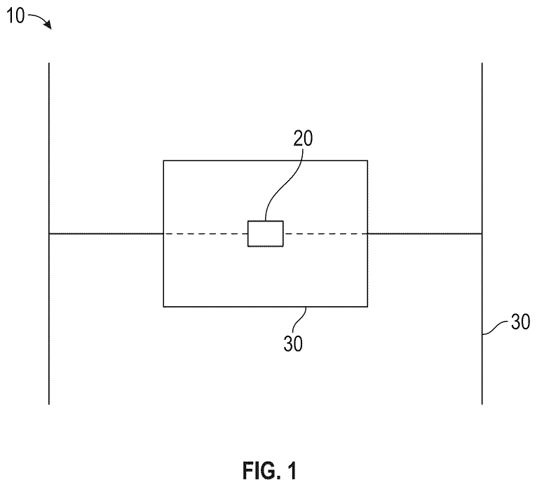

FIG. 1 illustrates a highly schematic satellite 10, or other space-based object, including an electronic system 20. In one example, the electronic system 20 is a camera imaging system. In alternative embodiments, the electronic system 20 can be communications systems, data collection systems, or any other electronic systems 20 suitable for operation in space-based applications. While illustrated with a single electronic system 20 in the schematic representation, it is appreciated that practical space-based applications such as the satellite 10 may include multiple distinct electronic systems 20, including power storage systems, power distribution systems, solar generation systems, and/or any number of other electronic systems configured to operate cooperatively or independently.

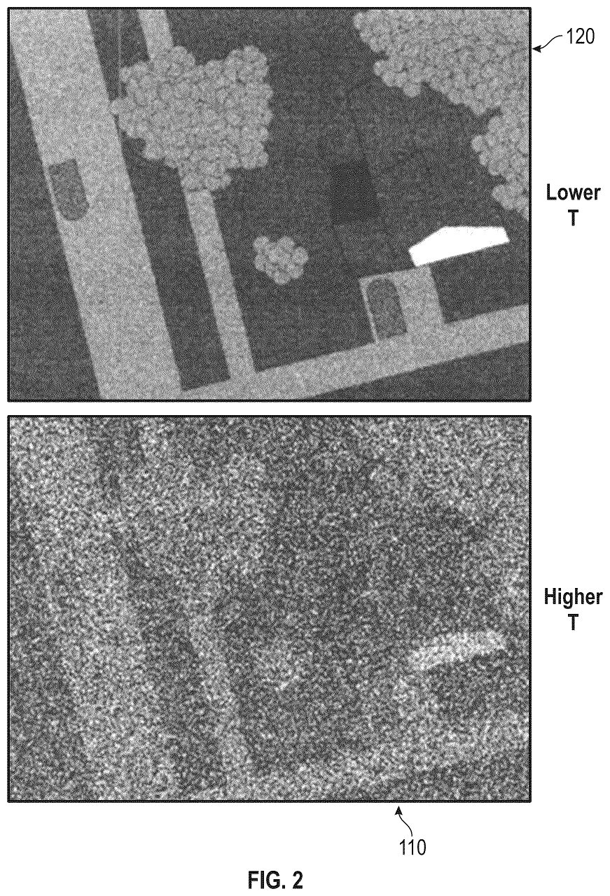

Operation of the electronic systems 20 generates waste heat which increases electronic noise and impacts the operation of the electronic systems 20. One such example is illustrated in FIG. 2, which illustrates a first image 110 generated by an electronic imaging system, such as the electronic system 20 at a higher temperature (e.g., approximately 68° F., 20° C.), and a second image 120 generated by the same electronic imaging system at a lower temperature (e.g., approximately −4° F., −20° C.). It is appreciated that practical implementations will be subjected to substantially different temperature ranges, and the illustrated and described ranges are nonlimiting and are provided for explanatory effect. By way of example, in some space applications the high and low temperatures may be −190.3° F., −123.5° C. to −154.3° F., −103.15° C.

Referring again to FIG. 1, absent a system for dissipating the waste heat, the heat accumulates within the satellite 10 and can cause sub-optimal operation of the electronic system(s) 20. Due to the lack of atmosphere, space-based applications emit heat radiatively from surfaces 30 of the satellite 10. However, such surfaces also generate a certain amount of heat due to light absorption. The surfaces 30 used to dissipate heat are referred to as “heat dissipation surfaces”. Heat dissipation surfaces are designed in such a manner that the surface can radiate heat, while simultaneously minimizing sunlight absorption thereby maximizing a total heat dissipation from the surface 30. The balancing act between heat radiation and light absorption is designed to maintain the electronics 20 to at the lowest possible temperature given the conditions.

To achieve the best available balance, existing heat dissipation surfaces typically utilize either quartz based tiles which are heavy, fragile, and cannot be applied to curved surfaces or use polymer foils, which have a substantially limited life cycle when exposed to the conditions typically found in space based applications.

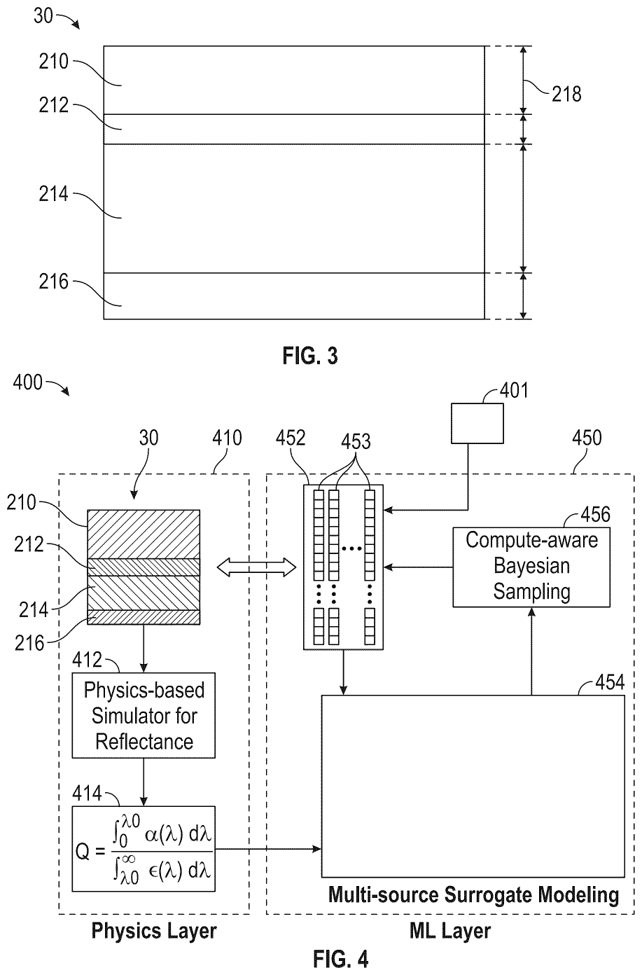

With continued reference to FIGS. 1-2, FIG. 3 illustrates a side view of an exemplary heat dissipation surface 30 of the satellite 10. The heat dissipation surfaces 30 are constructed of multiple layers 210, 212, 214, 216, with an outermost layer 210 being a layer of the heat dissipation surface 30 facing outward and exposed to space. The combined structure and composition of all the layers 210, 212, 214, 216 is referred to as the “metamaterial”. Each layer 210, 212, 214, 216 has a thickness 218 and a distinct material composition. Alternative example heat dissipation surfaces can include more or less layers 210, 212, 214, 216, and each layer 210, 212, 214, 216 can have more or less thickness. By selecting distinct materials of designed thicknesses. The metamaterial can achieve a target balance between the emissivity of the surface 30 and a light absorption of the surface 30. However, even when limiting the number of layers 210, 212, 214, 216 and providing constraints on the possible materials and the thicknesses 218 of each layer 210, 212, 214, 216, the design complexity results in an extreme magnitude of possible metamaterial structures.

To provide scale of the design complexity involved in determining a suitable composition and structure of the heat dissipation surface 30, a 10-layer one dimensional metamaterial with six possible material compositions (the layers can each be composed of one of six candidate materials) can have more than 60 million possible configurations. This design space expands even further (beyond trillions) when the thicknesses of each of the 10 layers at each possible configuration are optimized between 5 nm and 1.5 μm (varied in 10 nm resolution) within overall thickness and weight constraints of the metamaterial.

Existing design systems either rely on expensive simulators to calculate metamaterial performance metrics and cannot exhaustively explore the large design spaces which include inter-dependent and continuous and categorical design variables within a limited computational system. Due to this, the current approaches have difficulty meeting spectrally-varying emissivity objectives, robustness, and/or meeting constraints on total thickness and mass of the surfaces, in light of the scale of the design complexity.

In order to address this difficulty, the process described herein utilizes an artificial intelligence (AI) thermal metamaterial design ecosystem in a data-efficient end-to-end manner utilizing a flexible machine learning (ML) representation to explore mixed (continuous and categorical) design spaces and large design spaces, multi-source surrogate modeling using simulations and compute-aware Bayesian sampling to reduce the number of expensive high-fidelity simulations required to determine a viable candidate design or set of viable candidate designs. Surrogate modeling is an engineering method used when an outcome of interest cannot be easily measured or computed, so an approximate and fast-inference mathematical/ML model of the outcome is trained from the data (existing or generated by simulation and experiments) used instead. Based on the surrogate predictions and uncertainties, the compute-aware Bayesian sampling module adaptively queries the physics-based simulator to generate data at high value design points and updates the surrogate model and iteratively converge to a design solution. The technique discussed herein reduces the number of expensive high-fidelity simulations compared to conventional design tools using a trade off between exploration and exploitation.

With continued reference to FIGS. 1-3, FIG. 4 illustrates a design ecosystem 400 for designing the combined structure and composition of a one to two dimensional thermal metamaterial 30 based on a received set of initial design constraints 401. The ecosystem 400 includes a physics layer 410 and a machine learning layer 450 (alternately referred to as an AI layer). In general operations, the physics layer 410 receives a metamaterial design 30 from a machine learning representation 452 of an expected suitable material design space and applies a first physics-based simulation 412 to determine a reflectance of the metamaterial 30. The reflectance determines how much light is absorbed by the material vs. how much is reflected by the material 30 and the reflectance is provided to a physics based equation 414.

The physics-based equation 414 determines a ratio (Q) of solar absorptivity to thermal emissivity. In one example, the equation 414 is:

$$Q = \frac{{\int }_{0}^{{λ}_{0}} α ( λ ) d λ}{{\int }_{{λ}_{0}}^{∞} ϵ ( λ ) d λ} ;$$

where ∫₀^{λ}^{₀}α(λ)dλ is an absorption of the metamaterial 30 over a portion of the spectrum representing the solar spectrum (0 to lambda 0), and ∫_{λ}_{₀}^{∞}ϵ(λ)dλ is an emission of the metamaterial 30. In alternate examples where the primary source of heat is not the sun, alternate limits on the integral could be utilized and may not be bounded below by zero or above by infinity. The practical bounds for a given application can be determined by one of skill in the art dependent on the expected operating environment and operations.

The ratio (Q) is provided to a multi-source surrogate modeling algorithm 454 within the machine learning layer 450. In addition to the determined ratio, the multi-source surrogate modeling algorithm receives a design input from the flexible machine learning representation 452. Based on these inputs, the multi-source surrogate modeling machine learning algorithm trains (updates at every such iterations) a fast inference mapping (a.k.a. surrogate model) between design input and design metrics (reflectance ratio) in an adaptive manner and provides a design objective mean and uncertainty prediction to a compute-aware Bayesian sampling module 456. Then an adaptive sample of a high value design point from the compute-aware Bayesian sampling module 456 is provided back to the machine learning representation 452 and the machine learning representation 452 is adapted with the new sampling data.

The layers 410, 450 within the AI-guided thermal metamaterial design ecosystem 400 are seamlessly interfaced to exchange function call/queries and data between the physics layer 410 and the machine learning layer 450. Given an initial design requirement 401, the machine learning layer 450 converts a one or two dimensional thermal metamaterial configuration to a series of arrays 453 indexed by material type and individual layer 210, 212, 214, 216 height. Each element at an appropriate index carries the thickness and other continuous properties of the corresponding layer 210, 212, 214, 216. The combined series of arrays 453 is the flexible machine learning representation 452 and enables a tractable exploration of mixed (continuous and categorical) design variable within a large design space. In one example, the continuous design variables are the thicknesses of the layers (e.g., a layer can be 10 nm thick or 10.1 nm or 10.01 nm, and possible values exist on a continuum). Categorical variables are the number of layers (a design can have 4 or 5 layers, but not 4.5) and the material assigned to each layer (it can be gold, or silica, but not a mixture of the two). In certain cases the composition of a layer could be a continuous variable: metals can have alloys, which are mixtures of different elements that can vary continuously (there are many types of stainless steel, which is an alloy of iron with chromium, nickel and carbon in different ratios) and analogous things can be achieved in dielectrics (e.g., silicon-rich silicon nitride and nitride-rich silicon nitride have different properties). In some practical implementations the materials are treated as categorical variables in order to simplify the problem statement.

In one example, the large design space can include between a number of possibilities on the scale of billions or trillions. Once the flexible machine learning representation 452 has been constructed, the multi-source surrogate model 454 is built by learning a fast inference mapping between design input and design metrics (the reflectance ratio Q) in an adaptive manner. The structure of multi-source surrogate models in the design ecosystem are configured such that their prediction accuracy does not degrade with increasing input dimensionality.

Based on the surrogate predictions and uncertainties, the compute-aware Bayesian sampling module 456 adaptively queries the physics-based simulator 412 to generate data at high value design points and update the surrogate model 454 and iteratively converge to a design solution. This technique reduces number of expensive high-fidelity simulations required in physics layer 410, compared to conventional design tools by a tradeoff between exploration and exploitation.

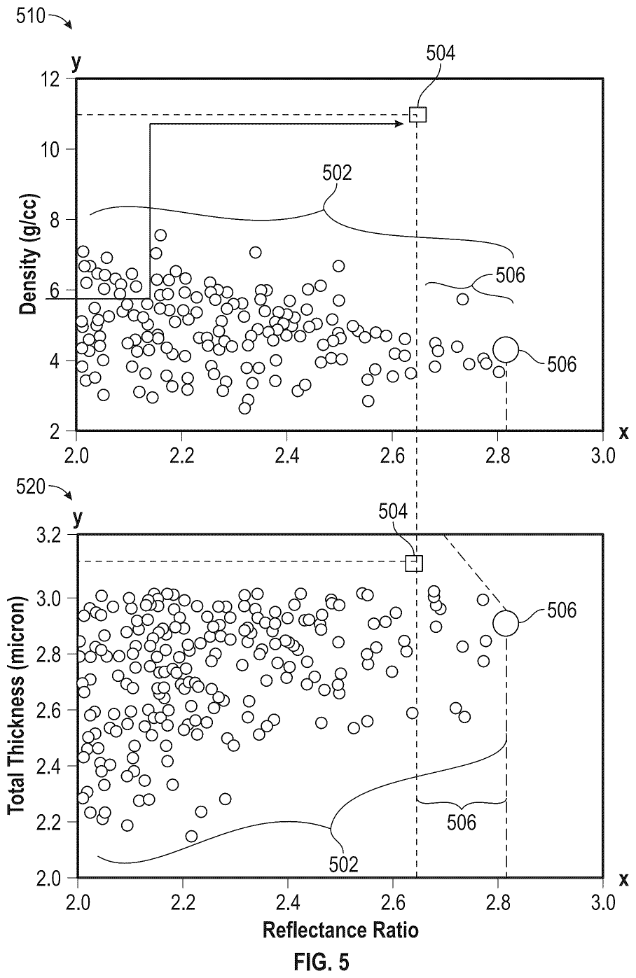

With continued reference to FIGS. 1-4, FIG. 5 illustrates application of the design ecosystem 400 of FIG. 4 to a five layer metamaterial design and generating a single candidate design. In alternate examples, additional candidate designs can be determined within the process. FIG. 5 includes a first chart 510 showing a density on a Y axis plotted vs. a reflectance ratio on an X axis and a second chart 520 showing a total thickness on a Y axis plotted vs. the reflectance ratio on an X axis. Each chart 510, 520 includes potential candidate materials 502 generated by the ecosystem 400 with a single “most suitable” metamaterial 506 that the ecosystem 400 has converged upon. Alongside the “most suitable” metamaterial 506, the set of determined materials 502 includes multiple suitable metamaterials 506 that could also be considered depending on various external factors including cost, ease of manufacturing, and the like. Also illustrated on each chart 510, 520 is a single suitable material 504 generated using a traditional method. The existing methods are limited to optimizing the thicknesses of 5 layers of a specific configuration, such as ‘silver’, ‘silica’, ‘titania’, ‘silica’, ‘titania’. The output of the existing methods is then fixed by a domain expert at the start of an existing adjoint-based optimization approach. In contrast, the ecosystem 400 doesn't require the limited starting assumption and it can explore through all possible configurations of the order of 6\hat{ }5 (=7776 for 5 layers with 6 material candidates at each layer) and optimize layer thicknesses simultaneously in approximately similar compute time as the existing method uses to optimize a specific configuration.

The suitable material generated using the traditional method has a reflectance ratio of approximately 2.65. Compared to this, multiple metamaterial designs output by the ecosystem 400 and meeting the design constraints provide better (higher) reflectance ratios. Furthermore, in addition to providing an improved reflectance ratio value for the metamaterial 30, the “most suitable” metamaterial 506 converged on by the design ecosystem 400 has a decreased density and a decreased total thickness relative to the traditional metamaterial 504. Both of these decreases represent an improvement on the nominal design on time of the decreased time required to identify the “most suitable” metamaterial 506.

In some examples, once the design ecosystem 400 has converged on a most suitable metamaterial 506, the computer system operating the design ecosystem can output the structure and composition of the most suitable metamaterial to a manufacturing system. The manufacturing system can then cause one or more heat dissipation surfaces 30 of the satellite 10 to be constructed of the metamaterial.

In some examples, the design ecosystem is configured as software modules stored within a single computer having a processor and a memory. In other examples, the design ecosystem can be software modules distributed throughout a network of computer systems, a cloud computing configuration, and/or any other distributed computing solution. As used herein, a computer system including a memory and a processor encompasses each of these possible variations without limiting the design ecosystem to a singular isolated physical computing device.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A computer assisted design ecosystem for materials design comprising:
a computer system including a memory and a processor, the memory storing a computer aided design ecosystem including a physics analysis module and a machine learning analysis module;
the physics module including a physics based reflectance simulation and a ratio of solar absorptivity to thermal emissivity module;
the machine learning module including a machine learning based surrogate model, a compute-aware Bayesian sampling module, and a machine learning metamaterial representation; and
wherein the memory and processor are configured to utilize the machine learning module and the physics module to iteratively determine a suitable metamaterial structure and composition in response to receiving at least one set of design constraints.

2. The computer assisted design ecosystem of claim 1, wherein the machine learning module is configured to receive the metamaterial representation and a calculated ratio of solar absorptivity to thermal emissivity, determine an adaptive sampling set, and provide the adaptive sampling set to the compute-aware Bayesian sampling module.

3. The computer assisted design ecosystem of claim 1, wherein the compute-aware Bayesian sampling module is configured to receive an adaptive sampling set, generate a machine learning representation of an expected suitable metamaterial design space based on the adaptive sampling set, and provide the machine learning representation of the expected suitable metamaterial design space to the physics module.

4. The computer assisted design ecosystem of claim 1, wherein the physics based simulator is configured to identify an example metamaterial structure and composition using a machine learning representation of an expected suitable metamaterial design space from the machine learning metamaterial representation and determine a reflectivity and an absorption rate of the example metamaterial structure.

5. The computer assisted design ecosystem of claim 4, wherein a ratio of solar absorptivity to thermal emissivity module of the example metamaterial structure and composition is determined using the ratio of solar absorptivity to thermal emissivity module, and wherein the ratio of solar absorptivity to thermal emissivity is provided to the multi-source surrogate modeling module.

6. The computer assisted design ecosystem of claim 1, wherein the computer system is further configured to output the suitable metamaterial structure and composition to a manufacturing system, and wherein the manufacturing system is configured to create the suitable metamaterial structure.

7. The computer assisted design ecosystem of claim 1, wherein the suitable metamaterial structure is a heat dissipation surface for a space-based unmanned component.

8. The computer assisted design ecosystem of claim 1, wherein utilizing the machine learning module and the physics module to iteratively determine a suitable metamaterial structure and composition in response to receiving at least one set of design constraints comprises iteratively determining a single most suitable metamaterial structure and composition.

9. The computer assisted design ecosystem of claim 1, wherein utilizing the machine learning module and the physics module to iteratively determine a suitable metamaterial structure and composition in response to receiving at least one set of design constraints comprises iteratively determining a plurality of suitable metamaterial structures and compositions, and wherein the machine learning based surrogate model is configured to output the plurality of suitable metamaterial structures and compositions to a material designer.

10. A method for generating a heat dissipation surface material for a space based application, the method comprising:
iteratively determining a suitable metamaterial structure and composition in response to receiving at least one set of design constraints using a computer based design ecosystem, the computer-based design ecosystem comprising a physics module including a physics based reflectance simulation and a ratio of solar absorptivity to thermal emissivity module and a machine learning module including a machine learning based surrogate model, a compute-aware Bayesian sampling module, and a machine learning metamaterial representation.

11. The method of claim 10, further comprising receiving the metamaterial representation and a calculated ratio of solar absorptivity to thermal emissivity at the machine learning module, determining an adaptive sampling set using the machine learning based surrogate model, and providing the adaptive sampling set to the compute-aware Bayesian sampling module.

12. The method of claim 10, further comprising receive an adaptive sampling set at the Bayesian sampling module, generate a machine learning representation of an expected suitable metamaterial design space based on the adaptive sampling set using the Bayesian sampling module, and providing the machine learning representation of the expected suitable metamaterial design space from the Bayesian sampling module to the physics module.

13. The method of claim 10, further comprising identifying an example metamaterial structure and composition using a machine learning representation of an expected suitable metamaterial design space from the machine learning metamaterial representation and determining a reflectivity and an absorption rate of the example metamaterial structure using the physics based reflectance simulation.

14. The method of claim 13, further comprising determining a ratio of solar absorptivity to thermal emissivity module of the example metamaterial structure and composition using the ratio of solar absorptivity to thermal emissivity module, and providing the ratio of solar absorptivity to thermal emissivity to the multi-source surrogate modeling module.

15. The method of claim 10, further comprising outputting the suitable metamaterial structure and composition to a manufacturing system and creating the suitable metamaterial structure.

16. The method of claim 15, further comprising constructing a space-based unmanned component including at least one heat dissipation surface comprising the suitable metamaterial structure.

17. The method of claim 10, wherein iteratively determining a suitable metamaterial structure and composition in response to receiving at least one set of design constraints comprises iteratively determining a single most suitable metamaterial structure and composition.

18. The method of claim 10, wherein iteratively determining a suitable metamaterial structure and composition in response to receiving at least one set of design constraints comprises iteratively determining a plurality of suitable metamaterial structures and compositions and outputting the determined plurality of suitable metamaterial structures to a materials designer.
